(19) United States
(12) Patent Application Publication
(10) Pub. No.: US 2023/0142077 A1
(43) Pub. Date: May 11, 2023

(54) COMPOSITION FOR PROMOTING ANGIOGENESIS USING LIQUID TYPE PLASMA AND METHOD FOR PROMOTING ANGIOGENESIS USING SAME

(71) Applicant: AJOU UNIVERSITY INDUSTRY-ACADEMIC COOPERATION FOUNDATION, Gyeonggi-do (KR)

(72) Inventors: Chul Ho KIM, Seoul (KR); Sung Un KANG, Gyeonggi-do (KR)

(73) Assignee: AJOU UNIVERSITY INDUSTRY-ACADEMIC COOPERATION FOUNDATION, Gyeonggi-do (KR)

(21) Appl. No.: 17/967,831

(22) Filed: Oct. 17, 2022

(30) Foreign Application Priority Data
Aug. 17, 2021 (KR) 10-2021-0108363

(51) Int. Cl. A61N 1/44

(52) U.S. Cl. CPC A61N 1/44

(57) ABSTRACT

The present invention relates to a method for preventing or treating angiogenesis-related diseases using a liquid type plasma. More specifically, the present invention relates to a method for preparing a liquid type plasma for preventing or treating angiogenesis-related diseases, a pharmaceutical composition for preventing or treating angiogenesis-related diseases using a liquid type plasma prepared by the method, and a method for preventing or treating angiogenesis-related diseases using the liquid type plasma.

### BACKGROUND

### 1. Field of the Invention

The present invention relates to a method for promoting angiogenesis using a liquid type plasma. More specifically, the present invention relates to a method for preparing a liquid type plasma for preventing or treating angiogenesis-related diseases, a pharmaceutical composition for preventing or treating angiogenesis-related diseases prepared using the liquid type plasma, and a method for preventing or treating angiogenesis-related diseases using the liquid type plasma.

The research of the present invention was supported by the Ministry of Health and Welfare (grant number: HR21C1003 (project unique number: 211003)).

### 2. Discussion of Related Art

Angiogenesis is a process of creating new blood vessels from existing blood vessels, and includes a migration process of vascular endothelial cells that constitute blood vessels, an invasion process of passing through the extracellular matrix (ECM), which is a barrier between cells, a process of proliferation of cells forming blood vessels, and a process by which blood vessels are formed (tube formation). This series of reactions is known to be tightly regulated by the balance between angiogenic factors and angiogenesis inhibitors (Schott R J and Morrow L A, Cardiovasc. Res., 1993, 27, 1155-1161). Such angiogenesis reactions are inhibited so as to hardly occur under normal conditions, and mainly occur during early embryonic development, wound healing and periodic changes in the female reproductive system. Except for the above cases, the replacement of blood vessels in the remaining part is usually slow, and endothelial cells of capillaries in most normal tissues can be seen to be in a quiescent state (Folkman J and Shing Y, J. Biol. Chem., 1992, 267, 10931-10934).

Vascular disease, particularly, ischemic disease, collectively refers to a disease in which various types of pathological abnormalities occur in blood vessels that supply blood to various organs of the body, resulting in local disturbance of normal blood flow.

Blood supply through blood vessels can be said to be an essential phenomenon for wound healing or tissue regeneration, and particularly, diseases such as arteriosclerosis, myocardial infarction and angina pectoris are caused by poor blood supply.

Further, angiogenesis must be involved in an essential wound healing process for regeneration of injured skin tissue. In the initial stage of a wound, an inflammatory response occurs due to necrosis of cells and destruction of blood vessels, and after the inflammatory response, the wound is subjected to a series of processes in which biological mediators such as kallikrein, thrombin, and plasmin are formed along with devascularization of blood components, activation of platelets and blood coagulation

Treatment of biological diseases using angiogenesis is called angiogenesis therapy, and angiogenic factors such as VEGF have already been used as therapeutic agents for severe local anemia. In addition, angiogenic factors such as FGF, epidermal growth factor (EGF) and platelet-derived endothelial growth factor (PDEGF) have also been studied for clinical treatment. However, the above factors are difficult to isolate and purify as proteins, and are expensive, thereby making clinical application difficult.

Meanwhile, low-temperature atmospheric pressure plasma is a plasma in a state where the energy possessed by electrons among the ions and electrons that constitute the plasma is greater than the energy possessed by the ions, and is also called low-temperature normal pressure plasma. That is, low-temperature plasma is produced as a result of changes in the outer electronic state of gas molecules caused by collision of electrons generated by a high-voltage electric discharge with the molecules of an exhaust gas during the discharge in the exhaust gas, and refers to a state in which radicals that are highly reactive chemically active species (for example, OH, COOH, CHO, and the like), excited molecules, and ions, which are positively or negatively charged to become an electrically neutral gas. Plasma is classified into a high-temperature thermal plasma in which electrons, ions, and molecules are all at high temperatures, and a low-temperature plasma in which only the electron temperature is high. Because the high-temperature thermal plasma can obtain high temperature and thus is used to melt materials and the low-temperature plasma has only high electron temperature, the low-temperature plasma has an advantage in that it can be applied to materials and conditions where high temperature cannot be applied and devices for it are simple. Since such a plasma causes various chemical reactions, many studies for utilizing the plasma and applying the plasma to various fields have been conducted. However, there is no known method of deriving an angiogenesis-promoting effect using the same to date.

Accordingly, the applicants of the present invention have conducted continuous research to develop a new composition for promoting angiogenesis, which overcomes the aforementioned problems, thereby completing the present invention. The present invention relates to a method for preventing or treating angiogenesis-related diseases using a liquid type plasma, and since the liquid type plasma of the present invention has a remarkable effect of promoting angiogenesis of blood vessels, it is expected to be widely utilized in the prevention and treatment of angiogenesis-related diseases.

### SUMMARY OF THE INVENTION

The present invention has been devised to solve the aforementioned problems in the related art, and relates to a method for preparing a non-thermal plasma having an effect of promoting angiogenesis. The present invention provides a pharmaceutical composition having an effect of preventing and treating angiogenesis-related diseases by irradiating a liquid material with the non-thermal plasma.

That is, an object of the present invention is to provide a method for preparing a liquid type plasma for preventing or treating angiogenesis-related diseases, a pharmaceutical composition for preventing or treating angiogenesis-related diseases prepared using the liquid type plasma, and a method for preventing or treating angiogenesis-related diseases using the liquid type plasma.

However, the technical problems to be achieved by the present invention are not limited to the aforementioned problem, and other problems that are not mentioned may be clearly understood by those with ordinary skill in the art from the following description.

Hereinafter, various exemplary embodiments described in the present application will be described with reference to drawings. In the following description, various specific details, for example, specific forms, compositions, processes, and the like are set forth for a thorough understanding of the present invention. However, specific exemplary embodiments may be practiced without one or more of these specific details, or in combination with other known methods and configurations. In other instances, known processes and preparation techniques have not been described in particular detail in order not to unnecessarily obscure the present invention. Reference throughout the present specification to “one exemplary embodiment” or “an exemplary embodiment” means that a particular feature, configuration, composition, or characteristic described in connection with the exemplary embodiment is included in at least one exemplary embodiment of the present invention. Therefore, the context of “in one exemplary embodiment” or “an exemplary embodiment” expressed in various positions throughout the present specification does not necessarily represent the same exemplary embodiment of the present invention. Additionally, the particular features, configurations, compositions, or characteristics may be combined in any suitable manner in one or more exemplary embodiments.

As used herein, the term “angiogenesis” may refer to all phenomena in which blood vessels are newly generated as a phenomenon in which blood vessels are generated, may refer to, for example, a phenomenon in which cells constituting existing blood vessels proliferate and migrate to create new blood vessels, and may also include a phenomenon in which blood vessels are newly generated by progenitor cells that create blood vessels.

In the present invention, “promoting angiogenesis” means that blood vessels such as capillaries are generated within a short period of time and/or more blood vessels are generated by promoting the angiogenesis as described above.

As used herein, “angiogenesis-related disease” refers to a disease that occurs or worsens when blood and oxygen are not properly supplied due to vascular abnormalities caused by various causes, and specifically refers to a disease that requires angiogenesis, but is not limited thereto. As an example, the angiogenesis-related disease may be at least one disease selected from the group consisting of wounds, burns, varicose veins, ischemia, infertility, diabetic foot ulcers, ischemic stroke, ulcers, arteriosclerosis, myocardial infarction, angina pectoris, ischemic heart failure, bedsores, alopecia and cerebrovascular dementia, but is not limited thereto.

In an exemplary embodiment of the present invention, “non-thermal atmospheric pressure plasma” refers to an ionized gas satisfying Debye shielding. Plasma may be considered as another state along with the three basic states of matter, gas, liquid, and solid, and is expressed as a fourth state. With regard to the plasma according to the present invention, a neutral gas experiences a phase transition to a plasma due to an external voltage, the excitation and ionization of the neutral gas may generate electrons and positive ions, and radicals in an excited molecular gas may be present. As the plasma generator, any known plasma generator may be used without limitation, as long as it can generate an atmospheric pressure plasma according to the objects of the present invention, and preferably, nitrogen gas is used, but the present invention is not limited thereto.

In an specific exemplary embodiment of the present invention, “liquid type plasma (LTP)” refers to generating a high-density and high-energy plasma in a liquid, and may be prepared by exposure to normal temperature non-thermal plasma (NTP) at atmospheric pressure. The term “liquid type plasma” can be used interchangeably with the term “plasma-conditioned liquid material,” and for the term “liquid material,” a material in the form of a liquid can be used without limitation, and is preferably water, saline, a buffer or a culture medium, and most preferably a medium.

Since the liquid type plasma of the present invention can be supplied in the form of a liquid composition, the liquid type plasma has an advantage in that it convenient to distribute and carry, there is less cell damage than directly treating a target body with plasma, there is no risk of skin damage such as burns due to the user's wrong operation of a device, and the liquid type plasma of the present invention can be uniformly applied even to a wide and curved area.

In an exemplary embodiment of the present invention, “culture medium” refers to a culture medium capable of supporting cell growth and survival in vitro, and includes all typical media used in the art suitable for culturing cells. According to the type of cell, culture media and culture conditions may be selected. The basal culture medium used for the culture of cells is preferably a cell culture minimum medium (CCMM), and generally includes a carbon source, a nitrogen source, and a trace element component. Examples of such a cell culture basal culture medium include Dulbecco's Modified Eagle's Medium (DMEM), Minimal Essential Medium (MEM), Basal Medium Eagle (BME), RPMI1640, F-10, F-12, Minimal Essential Medium, Glasgow's Minimal Essential Medium (GMEM), Iscove's Modified Dulbecco's Medium, and the like, but are not limited thereto.

In an exemplary embodiment of the present invention, “treatment” refers to any act of ameliorating or beneficially altering the symptoms of angiogenesis-related diseases or diseases caused thereby using the liquid type plasma according to the present invention. A person with ordinary skill in the art to which the present application pertains can refer to the materials presented by the Korean Medical Association, and the like to understand the accurate criteria for angiogenesis-related diseases and judge the degree of alleviation, amelioration and treatment.

In an exemplary embodiment of the present invention, “prevention” refers to any act of inhibiting or delaying the onset of angiogenesis-related diseases or other diseases caused thereby using the liquid type plasma according to the present invention. It will be obvious to those skilled in the art that the composition of the present application, which has a therapeutic effect on angiogenesis-related diseases, can prevent such diseases using the liquid type plasma according to the present invention before the initial symptoms or symptoms of angiogenesis-related diseases appear.

As used herein, “pharmaceutical composition” refers to a composition that is administered for a specific purpose. For the purposes of the present invention, the pharmaceutical composition of the present invention includes a liquid type plasma prepared by irradiating a liquid material with plasma as an active ingredient, and may include a protein involved in this process, and a pharmaceutically acceptable carrier excipient or diluent. The aforementioned “pharmaceutically acceptable” carrier or excipient refers to those that are approved by a governmental regulatory authority or listed in governmental or other generally approved pharmacopoeia for use in vertebrates and more particularly in humans.

For parenteral administration, the pharmaceutical composition of the present invention may be in the form of a suspension, solution or emulsion in an oily or aqueous carrier, and may be prepared in the form of a solid or semi-solid. In addition, the pharmaceutical composition of the present invention may include a formulating agent such as a suspending agent, a stabilizer, a solubilizer, and/or a dispersant, and may be sterilized. The pharmaceutical composition may be stable under preparation and storage conditions, and may be preserved against the contaminating action of microorganisms such as bacteria or fungi. Alternatively, the pharmaceutical composition of the present invention may be provided in a sterile powder form for reconstitution with a suitable carrier prior to use. The pharmaceutical composition may be provided in a unit-dose form, or may be present in microneedle patches, in ampoules, or in other unit-dose containers, or in multi-dose containers. Alternatively, the pharmaceutical composition may be stored in a sterile liquid carrier, for example, in a freeze-dried state that requires only the addition of water for injection just prior to use. Immediately injectable solutions and suspensions may be prepared from sterile powders, granules, or tablets.

In some non-limiting exemplary embodiments, the pharmaceutical composition of the present invention may be formulated or included in the form of microspheres in a liquid. In certain non-limiting exemplary embodiments, the pharmaceutical composition of the present invention may include a pharmaceutically acceptable compound and/or mixture thereof at a concentration of 0.001 to 100,000 U/kg. Furthermore, in certain non-limiting exemplary embodiments, a suitable excipient of the pharmaceutical composition of the present invention may include a preservative, a suspending agent, an additional stabilizer, a dye, a buffer, an antibacterial agent, an antifungal agent, and an isotonic agent, for example, sugar or sodium chloride. As used herein, the term “stabilizer” refers to a compound optionally used in the pharmaceutical composition of the present invention to increase shelf life. In a non-limiting exemplary embodiment, a stabilizer may be a sugar, an amino acid, or a polymer. Further, the pharmaceutical composition of the present invention may include one or more pharmaceutically acceptable carriers, and the carrier may be a solvent or a dispersion medium. Non-limiting examples of the pharmaceutically acceptable carrier include water, saline, ethanol, polyols (for example, glycerol, propylene glycol, and liquid polyethylene glycol), oils, and suitable mixtures thereof. Non-limiting examples of a sterilization technique applied to the pharmaceutical composition of the present invention include filtration through a bacteria-inhibiting filter, terminal sterilization, incorporation of sterile preparations, irradiation, sterilization gas irradiation, heating, vacuum drying, and freeze drying.

In the present specification, “administration” means introducing the composition of the present invention to a patient by any appropriate method, and for the route of administration of the composition of the present invention, the composition of the present invention may be administered by any general route as long as it can reach a target tissue. The pharmaceutical composition of the present invention may be applied through oral administration, intraperitoneal administration, intravenous administration, intramuscular administration, subcutaneous administration, intradermal administration, intranasal administration, intrapulmonary administration, intrarectal administration, intraluminal administration, intraperitoneal administration, or intradural administration, but the pharmaceutical composition of the present invention is most preferably provided in the form of being applied to the skin or through subcutaneous or intradermal injection, but the present invention is not limited thereto.

The treatment method of the present invention may include administering the pharmaceutical composition in a pharmaceutically effective amount. An effective amount in the present invention may be adjusted according to various factors such as type and severity of a disease, types and contents of an active ingredient and other ingredients contained in the composition, type of a dosage form, age, body weight, general medical conditions, gender and diet of a patient, duration and route of administration, a release rate of the composition, treatment duration, and the number of drugs simultaneously used.

An agent for preventing or treating angiogenesis-related diseases such as wounds, burns, varicose veins, ischemia, infertility, diabetic foot ulcers, ischemic stroke, ulcers, arteriosclerosis, myocardial infarction, angina pectoris, ischemic heart failure, bedsores, alopecia and cerebrovascular dementia according to the present invention may be administered daily or intermittently, and the administration frequency per day can be 1 time or divided into 2 to 3 times. Further, the compositions of the present invention can be used alone or in combination with other drug treatments for the prevention or treatment of angiogenesis-related diseases. It is important to administer the composition in a minimum amount that can obtain the maximum effect without any side effects, in consideration of all the aforementioned factors, and this amount may be easily determined by those skilled in the art.

In an exemplary embodiment of the present invention, the method for preparing a liquid type plasma for promoting angiogenesis may include (a) filling a plasma generator with a carrier gas, (b) generating plasma by supplying a voltage of 1 kV to 10 kV and a frequency of 10 to 30 kHz to the plasma generator and (c) irradiating a liquid material with the generated plasma. In the present exemplary embodiment, the carrier gas in Step (a) may be selected from the group consisting of nitrogen, helium, argon, and oxygen, and is most preferably nitrogen, but is not limited thereto.

According to an exemplary embodiment of the present invention, the irradiation in Step (c) may be performed for 5 minutes to 120 minutes, and the liquid material in Step (c) may be water, saline, a buffer, or a medium.

The present invention also provides a pharmaceutical composition for preventing or treating angiogenesis-related diseases, including the liquid type plasma prepared by any one method of the methods as an active ingredient.

According to an exemplary embodiment of the present invention, the pharmaceutical composition may be prepared as an oral formulation, a parenteral formulation or a topical formulation, and the pharmaceutical compositions may be administered alone or in combination with methods using surgery, radiation therapy, hormone therapy, chemotherapy and a biological response modifier.

The present invention also provides a method for preventing or treating angiogenesis-related diseases, the method including administering a liquid type plasma prepared by any one method among the method for preparing a liquid type plasma to a subject.

Specifically, the method for preventing or treating angiogenesis-related diseases may be applied to a disease selected from the group consisting of wounds, burns, varicose veins, ischemia, infertility, diabetic foot ulcers, ischemic stroke, ulcers, arteriosclerosis, myocardial infarction, angina pectoris, ischemic heart failure, bedsores, alopecia and cerebrovascular dementia, and may be preferably applied for skin flap regeneration, wound and burn treatment, artificial skin grafting, and blood vessel production for grafting.

It is preferred that a therapeutically effective amount of a liquid type plasma for treating angiogenesis-related diseases of the present invention is differently applied depending on various factors including the type and extent of a response to be achieved, a specific composition, including whether other formulations are used according to the case, the age, body weight, general health status, gender, and diet of a subject, the time of administration, the route of administration, the secretion rate of the composition, the period of treatment, a drug used in combination with or concurrently with the specific composition and similar factors well known in the medical field. Therefore, it is preferred that the effective amount of the composition suitable for the purpose of the present invention is determined in consideration of the above-described details.

The subject can be applied to any mammal, and the mammal includes a human and a primate, as well as livestock such as cows, pigs, sheep, horses, a dogs and a cats.

Furthermore, the present invention provides a use of a liquid type plasma prepared to produce a pharmaceutical preparation having an effect of preventing or treating wounds or infectious diseases.

Hereinafter, the present invention will be described in detail step by step.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1A is a schematic view of a plasma apparatus and an experimental configuration according to an exemplary embodiment of the present invention. FIG. 1B is an image illustrating the optical emission spectrum at atmospheric pressure by a plasma source using 5 L/min of N₂ gas. FIG. 1C illustrates the voltage and current waveforms of a plasma jet using 5 L/min of N₂ gas. Referring to FIG. 1C, it can be seen that a waveform of a general pen-type plasma jet with one current peak occurring for each half cycle appears. FIG. 1D illustrates a time-concentration graph of ozone produced by a plasma jet measured by an ozone analyzer according to an exemplary embodiment of the present invention at atmospheric pressure. FIG. 1E is a time-concentration graph of NO and NO₂ at atmospheric pressure for a plasma jet measured by a gas analyzer. FIG. 1F illustrates a water temperature versus plasma treatment time graph of a N₂ plasma-treated solution, and FIG. 1G is a graph showing time-pH values measured during plasma treatment.

FIG. 2 illustrates the results of comparing a group treated with N₂ plasma according to the present invention and a control (untreated group). A non-thermal plasma treated solution (NTS) induces endothelial cell migration, which is proven by a Matrigel plug analysis which induces angiogenesis. FIG. 2A illustrates the viability of endothelial cells upon N₂ plasma treatment according to the present invention. In FIG. 2A, there was no statistical significance between a treated group and an untreated group (N=10, each group). FIG. 2B illustrates the results of analyzing a Matrigel plug according to an exemplary embodiment of the present invention, and is an image confirming angiogenesis in response to the injection of Matrigel into endothelial cells. Compared to the control in FIG. 2B, it can be seen that capillary tube formation becomes thicker in an NTS-treated group (Scale bar=20 μm). FIG. 2C is an image confirming the morphology of Matrigel plugs harvested from NTS-treated mice and control mice (Scale bar=20 μm). FIG. 2D is a set of images observed after H&E staining of Matrigel plugs of control and NTS-treated mice (Scale bar=200 μm). FIG. 2E is a set of images of observing the immunofluorescence analysis results of an endothelial cell marker CD31 (red) and nuclei labeled with DAPI (blue) in Matrigel plug sections of control and NTS-treated mice (Scale bar=100 μm). The bottom of FIG. 2E illustrates a graph of the quantification (10 fields/group) of CD31-positive cells (***p<0.001).

FIG. 3 illustrates the results of confirming the detection of intracellular nitric oxide by NTS, showing the effects of eNOS phosphorylation and angiogenesis. In FIG. 3A, cell proliferation was measured by BrDu assay, and NTS increased HUVEC cell proliferation. Statistically significant cell proliferation was increased with NTS treatment time (**P<0.01; NS=not significant). FIGS. 3B and 3C illustrate the results of analyzing intracellular nitric oxide (NO), which was analyzed by flow cytometry (FIG. 3B) and a fluorescence image (FIG. 3C) using DAF-FM probes in control and NTS-treated cells, respectively. Bar graphs in FIG. 3 indicate the mean±standard deviation of each independent experiment (***P<0.001). FIG. 3 also confirms the simulated effect of NTS on HUVEC tube formation. FIGS. 3D and 3E illustrate images of the results after NTS treatment and culture for 6 hours. In FIGS. 3D and 3E, cells cultured under control conditions or cultured after NTS treatment were seeded onto Matrigel and then stained with Calcein-AM and fluorescently examined (Scale bar=1000 μm). In the corresponding drawings, tube formation was quantified, and ImageJ plug-in software was used to determine the overall lengths of tube-like structures in the images. Histograms show tube formation as a percentage of control cells (N=5, ***P<0.001).

eNOS signaling is involved in the NTS-induced angiogenic pathway. In FIGS. 3F and 3G, immunoblotting was performed on phosphorylated eNOS (s1177), and it can be seen that NTS induced angiogenesis in a dose- and time-dependent manner with phosphorylated eNOS (S1177).

FIG. 4 illustrates the results of confirming the effect of NTS on the improvement of eNOS-activated growth and capillary structure formation in HUVECs according to an exemplary embodiment of the present invention. FIG. 4 analyzes the effect of NTS on cell proliferation by BrDu assay after treatment of HUVECs several times (30, 60 sec/ml) with NTS in order to examine the effect of NTS on endothelial cells, and referring to FIG. 4A, levels of extracellular nitric oxide (NO), which is the RNS, were increased in the presence of NTS, whereas ROS levels were not significantly different compared to cells not treated with NTS in FIGS. 4B and 4C.

FIG. 5 illustrates the results of experiments confirming that endothelial cell migration and ECM production are enhanced by NTS. FIG. 5A is an image confirming the effect of NTS treatment on the migratory potential of HUVECs, which was analyzed through wound migration analysis. Confluent cells were wounded using a p1000 pipette tip, and the control was subjected to no treatment. Thereafter, they were treated with NTS for 24 hours. Wound migration analysis shows that NTS treatment promotes endothelial cell migration for 30 and 60 seconds. An average denuded zone was obtained by calculating the ratio of the average area of the denuded zone to the area of the control. In the corresponding drawing, asterisks denote a statistically significant difference (***P<0.001).

FIG. 5B is an image confirming whether NTS regulates the total protein expression of VE-cadherin and ECM (p-FAK(Y397), FAK, p-Src (Y418), Src) molecules using western blotting. FIG. 5B illustrates the results of treating HUVEC cells with NTS for 30 seconds and 60 seconds, and then culturing the HUVEC cells for 24 hours, and α-tubulin was used as a loading control. At the bottom of FIG. 5B, the band intensities according to the above experiments were measured and are shown graphically (*P<0.05, **P<0.01).

FIG. 5C is a zymogram showing that an increase in concentrations of NTS is associated with a selective increase in MMP2 activity according to an exemplary embodiment of the present invention. 72 KDa and 62 KDa MMP activity bands were quantified (expressed as a proportion of the control) in FIG. 5C (**P<0.01, ***P<0.001).

FIG. 5D illustrates the results of confirming the relative expression levels of mRNA of MMP2 and MMP9 by real-time PCR. Referring to FIG. 5D, it can be seen that no significant change in MMP-9 activity is evident. FIGS. 5E and 5F illustrate immunocytochemical analysis results for VE-cadherin and p-FAK according to an exemplary embodiment of the present invention. Referring to FIG. 5E, VE-cadherin was reduced in NTS-treated cells (Scale bar=20 pin). In FIG. 5F, after NTS treatment, FAK local accumulation was significantly increased in NTS-treated cells (Scale bar=30 pin).

An NOS inhibitor (L-NMMA, 1 mM) may selectively regulate eNOS expression, angiogenesis and migration. FIG. 6A illustrates the results of analyzing the expression and phosphorylation of total eNOS by western blot according to an exemplary embodiment of the present invention. According to FIG. 6A, the phosphorylation of eNOS was upregulated after NTS treatment and significantly attenuated by L-NMMA. The graph on the right of FIG. 6A is a graphical representation of the band intensities measured by western blot (***P<0.001). FIG. 6B illustrates a flow cytometry result using a DAF-FM probe in cells treated with L-NMMA according to an exemplary embodiment of the present invention. The bar graph in the bottom of FIG. 6B indicates the mean±standard deviation of three independent experiments (***P<0.001). FIG. 6C is an image confirming angiogenic activity in HUVEC cells treated with NTS and L-NMMA for analysis of tube formation according to an exemplary embodiment of the present invention (Scale bar=1000 μM). The bottom of FIG. 6C is a bar graph for the quantification of tube formation, and N=5, ***P<0.001.

FIG. 6D illustrates the results of analyzing wound migration according to an exemplary embodiment of the present invention. FIG. 6D confirms the migration potential of cells by treating HUVEC cells with NTS and L-NMMA. The bottom of FIG. 6D is a bar graph for the quantification of cell migration (***P<0.001, NS=not significant. Scale bar=1000 μM).

FIG. 6F illustrates the zymogram analysis results according to an exemplary embodiment of the present invention. In FIG. 6F, NTS-activated MMP2 was significantly attenuated by L-NMMA, and the band intensities of the corresponding results were measured and are shown graphically in the bottom of FIG. 6F (***P<0.001, NS=not significant).

FIG. 6G is an image confirming the expression of VE-cadherin after treatment with NTS, L-NMMA and NTS+L-NMMA according to an exemplary embodiment of the present invention. Referring to FIG. 6G, it can be seen that the expression of VE-cadherin was reduced after NTS treatment and was significantly attenuated by L-NMMA (Scale bar=100 μm).

FIG. 7 is a graph showing the extent of cell proliferation after treatment with NTS and L-NMMA according to an exemplary embodiment of the present invention.

FIG. 8 confirms that NTS-induced eNOS signaling mediates phosphorylation of LKB1/AMPK. FIGS. 8A and 8B illustrate the western blot results of time course experiments analyzed on cells stimulated with NTS under each condition. In FIGS. 8A and 8B, cell lysates are used to determine the phosphorylation of AMPK and LKB1. The corresponding results are shown graphically at the bottom of FIGS. 8A and 8B by measuring the band intensity (***, P<0.001). LKB1/AMPK signaling regulates upstream NTS-induced angiogenesis and migration.

FIG. 8C illustrates western blot results according to an exemplary embodiment of the present invention. HUVECs were transfected with AMPK-siRNA (100 pmol) or control siRNA for 24 hours and then treated with NTS for 60 seconds. After 24 hours, cell lysates were analyzed by western blot using antibodies against p-AMPK, T-AMPK p-eNOS, T-eNOS, VE-cadherin, p-FAK, T-FAK, p-Src, and T-Src.

FIG. 8D is a set of images showing tube formation analysis results according to an exemplary embodiment of the present invention. Angiogenic activity in AMPK siRNA-transfected HUVEC cells was confirmed (Scale bar=1000 μM). The bottom of FIG. 8 is a bar graph quantifying the extent of tube formation (N=5, ***P<0.001).

FIG. 8E is a set of images showing wound migration analysis results according to an exemplary embodiment of the present invention. In FIG. 8E, the migration activity in AMPK siRNA-transfected HUVEC cells is confirmed, and the graph at the bottom quantifies and shows the extent of cell migration (***P<0.001, Scale bar=1000 μM).

FIGS. 8F and 8G illustrate the results of immunocytochemical analysis for VE-cadherin and p-FAK. In FIG. 8F, the expression of VE-cadherin was reduced upon NTS treatment, and the decrease in VE-cadherin expression was increased in cells transfected with AMPK siRNA (scale bar=20 μm). In FIG. 8G, the expression of p-FAK was attenuated in cells transfected with AMPK siRNA (Scale bar=30 μm).

FIG. 8H is a schematic view of the NTS-induced eNOS signaling process, and illustrates the phosphorylation of LKB1/AMPK.

### DETAILED DESCRIPTION OF EXEMPLARY EMBODIMENTS

Hereinafter, the present invention will be described in more detail through Examples. These Examples are provided only for more specifically describing the present invention, and it will be obvious to those with ordinary skill in the art to which the present invention pertains that the scope of the present invention is not limited by these Examples according to the gist of the present invention.

### Example 1. Preparation of Non-Thermal Liquid Type Plasma (NTS)

Plasma was prepared using an atmospheric pressure plasma generator (non-thermal plasma jet system) equipped with a nozzle case and a plasma generation module. The plasma generation module may be composed of a Ni—Co alloy electrode, a glass insulator and an electrode ring.

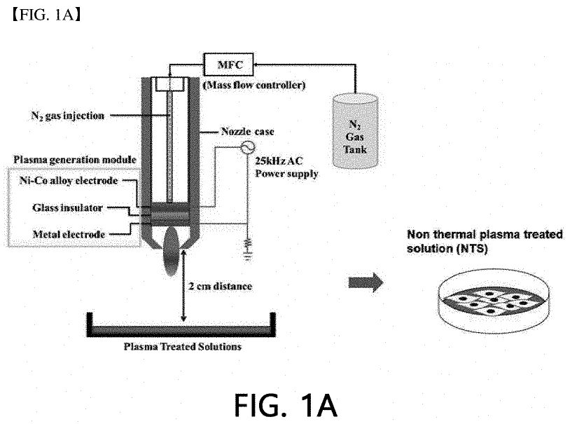

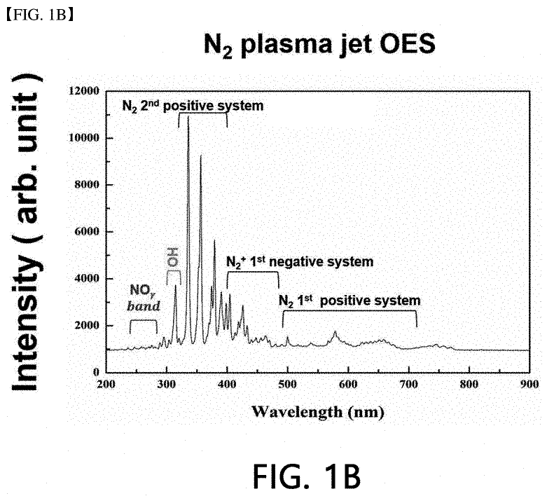

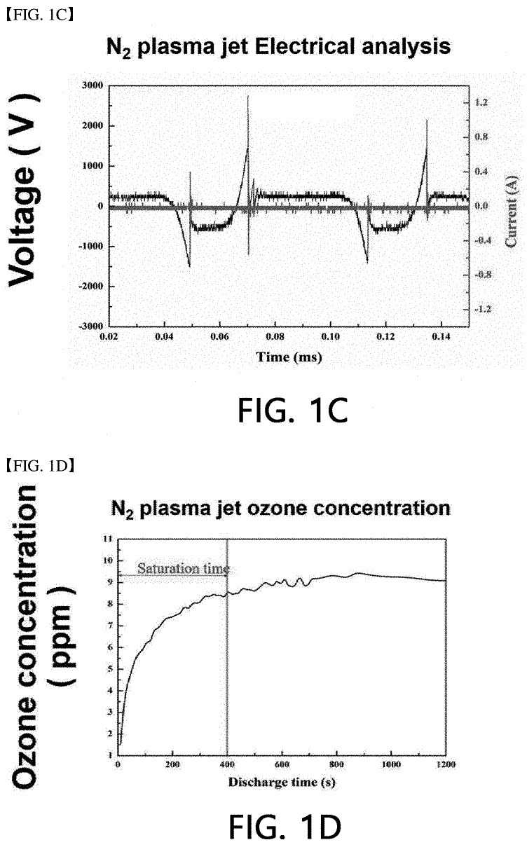

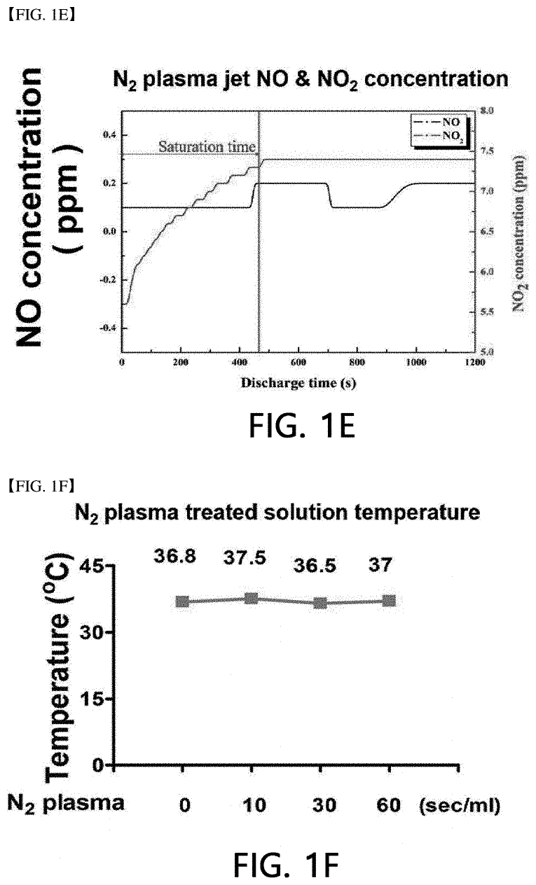

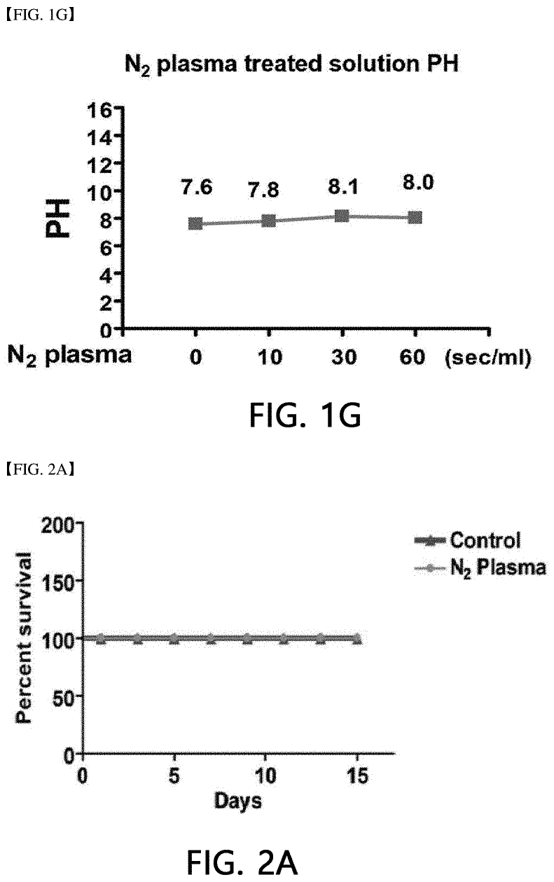

It is important to generate plasma while maintaining a low temperature so as not to damage the surface of a biological sample, and a dielectric barrier discharge (DBD) method was used for this purpose. In the plasma generator of the present invention, arcing was prevented by inserting a dielectric between the electrodes. The device has a gas delivery nozzle diameter of less than 3 mm and was designed to generate a 1-inch uniform plasma for medical research. A liquid type plasma was prepared by a method of supplying a carrier gas to the device at a flow rate of 10 (standard) L/min and treating a culture dish (12-well plate, TPP, Renner, Dannstadt, Germany) in which 2 ml of a cell medium was dispensed with plasma at a distance of 2 cm spaced from the bottom surface of the culture dish for 30 seconds per 1 ml. In this case, the power supply specifications of the plasma device are preferably a power of 1 to 20 kV and an average frequency of 1 to 10 kHz, most preferably a power of 3 kV and an operating frequency of 25 kHz, but are not limited thereto. A schematic view of the method for preparing a liquid type plasma is illustrated in FIG. 1A.

### Example 2. Non-Thermal Liquid Type Plasma (NTS) Intracellular Experiments

### Example 2-1. Cell Culture and NTP Treatment

Human umbilical vein endothelial cells (HUVECs, Lonza, UK) were purchased from Lonza (CC-2935, Cell Catalog, Lonza, UK). Cells were maintained in an endothelial growth medium (EGM-2, CC-3162, Lonza, UK) at 37° C. and 5% CO₂ under humidified conditions for proliferation, and HUVECs older than P6 were discarded because they lost their ability to form tubes.

Thereafter, 2 ml of a cell culture medium was added to a Petri dish (6 well plate, TPP, Z707767, Renner, Dannstadt, Germany) for NTS treatment. The distance between the plasma device and the bottom of the Petri dish was maintained to be about 2 cm, and the cells were treated with NTS for 30 and 60 seconds per ml.

### Example 2-2. Cell Proliferation Analysis

Cell proliferation was measured with Cell Proliferation ELISA, BrdU (colorimetric) (Roche Diagnostics, 11647229001, Penzberg, Germany). A known method was used for the cell proliferation analysis. The HUVEC cells cultured according to Example 2-1 were seeded in a 96-well cell culture plate at a density of 4×10³ cells/well, and after 24 hours, the cells were treated with NTS.

Cell proliferation results were expressed as a percentage of untreated cells set to 100%.

### Example 2-3. Intracellular NO Production Effect Analysis

Nitric oxide (NO) levels were confirmed by measuring fluorescence changes in 4-amino-5-methylamino-2′,7′-difluorofluorescein diacetate (DAF-FMDA, Thermo Fisher, D23844, Eugene, Oreg., USA) due to oxidation, respectively. Cells were cultured with reagents according to the manufacturer's instructions. Changes in DAF-FM fluorescence were measured by flow cytometry (BD Biosciences) and a fluorescence microscope (EVOS FL Auto, Thermo Fisher) after 24 hours. NG-methyl-L-arginine acetate salt (L-NMMA, Sigma, M7033) was used to suppress the production of nitric oxide (NO).

### Example 3. Non-Thermal Liquid Type Plasma (NTS, Non-Thermal Plasma Treated Solution) Extracellular Experiments

### Example 3-1. Tube Formation Analysis

HUVEC cells were trypsinized and then seeded onto a 96-well plate (2×10⁴/well) pre-coated with 40 μl (10 mg/ml) of growth factor-reduced Matrigel (BD Biosciences, Billerica, Mass.) in an EBM2 medium.

After incubation at 37° C. for 30 minutes, 1 hour, 3 hours, and 6 hours, viable cells were detected by staining with Calcein AM (Trevigen, Gaithersburg, Md.), and then a capillary-like structure was imaged using EVOS FL Auto (ThermoFisher). Data in the images was quantified with National Institutes of Health (NIH) ImageJ 1.41q software.

### Example 3-2. Western Blot

Western blot was performed using a known method. Cells were lysed in a RIPA buffer (Sigma Aldrich) including 150 mM NaCl, 1.0% Nonidet-P 40, 0.5% sodium deoxycholate, 0.1% sodium dodecyl sulfate, 50 mM Tri (pH 8.0), a protease inhibitor cocktail and PhoSTOP (Roche Molecular Biochemicals, Basel, Switzerland).

A primary antibody used in the present experiment was purchased from Cell Signaling (Danvers, Mass., USA) and VE-cadherin, p-LKB1, and LKB1 were purchased from Santa Cruz (Cambridge, UK). A secondary antibody (anti-rabbit IgG or anti-mouse IgG, 1:2000) was purchased from Cell Signaling Technology.

Data in the images was quantified with National Institutes of Health (NIH) ImageJ 1.41q software.

### Example 3-3. Immunocytochemical Analysis

Immunocytochemical analysis was performed using a known method. The cells cultured according to Example 2-1 were additionally cultured on coverslips (Thermo Fisher Scientific, Rochester, N.Y., USA) and treated with NTS (60 sec/ml) or a vehicle control.

The coverslips were then treated with polyclonal rabbit anti-LC3B, p-FAK, and VE-Cadherin (1:200; Cell Signaling Technology, Danvers, Mass., USA), incubated for 2 hours, washed with PBS, and then incubated with an Alexa 488-labeled antibody for 1 hour.

Thereafter, after being washed three times with PBS, the slides were stained with Hoechst 33258 (Molecular Probe), and phalloidin (1:50; Molecular Probes, R415) was added thereto for 15 minutes to counterstain nuclei and F-actin. The stained coverslips were washed with PBS, mounted with Vectashield (Vector Laboratories, Inc., Burlingame, Calif., USA), and then analyzed using EVOS FL Auto (Thermo Fisher).

### Examples 3-4. Wound Healing Analysis

Wound healing analysis was performed using a known method. A monolayer of 100% confluent cells was scratched with a sterile pipette tip (1 ml) and washed extensively to remove cell debris. Thereafter, the remaining cells were treated with NTS and incubated at 37° C. for 24 hours.

The results of the present experiment were automatically recognized and measured by Metamorph® NX image software (Molecular Devices, Sunnyvale, Calif., USA), and a crystal violet (cat) staining eluate was measured under an optical microscope (EVOS FL Auto) and then illustrated as an image.

### Example 3-5. Gelatin Zymogram Analysis

Matrix metalloproteases were analyzed using a known gelatin zymography method. The cells according to Example 2-1 were cultured in a 6-well plate (Corning Scientific, Rochester, N.Y., USA) and treated with NTS (60 sec/ml) or a vehicle control. Thereafter, the supernatant (100 μl) of each sample was mixed with 1 μl of 100 mM 4-aminophenylmercuric acetate (Sigma-Aldrich), and the sample was incubated at 37° C. for 1 hour. After each sample was placed in a sample buffer (excluding 2-mercaptoethanol) for 10 minutes, the samples were electrophoresed on an a 8% polyacrylamide gel containing 1% gelatin.

The gel was incubated at room temperature for 60 minutes in a regeneration buffer, and then incubated in 100 ml of a development buffer at 37° C. with gentle shaking. Thereafter, the gel was stained with Coomassie Brilliant Blue for 3 hours. After destaining with 400 ml of a destaining solution (methanol, 100 ml of acetic acid, 500 ml of distilled water), images were obtained using an image analyzer.

### Example 3-6. Quantitative Real-Time PCR

Quantitative real-time PCR analysis was performed according to a known method. A target gene was quantified by one-step real-time PCR using StepOnePlus™ (Applied Biosystems, Foster City, Calif.). qPCR primers were purchased from Qiagen (Qiagen, Germantown, Md., USA), and GAPDH mRNA levels were used for normalization.

### Example 3-7. Small Interference RNA Transfection

In the present experiment, transient transfection was performed using the Lipofectamine 2000 reagent (Thermo Fisher Scientific). siRNA was obtained from Santacruz (Santa Cruz, Calif., USA).

### Example. 3-8. Matrigel Plug Analysis

Matrigel (BD Biosciences) with or without NTS was injected subcutaneously into the right flank of C57/BL6 male mice (Kostech Co.) at a dose of 400 respectively. After 15 days, the mice were sacrificed, the hard Matrigel plugs were carefully removed without the surrounding connective tissue, and then photographs were taken.

The number of endothelial cells in each plug was evaluated by immunostaining with a CD31 antibody (1:500 dilution).

### Example 3-9. Statistical Analysis

Data parameters in the present specification are expressed as mean±standard deviation (SD).

In each analysis, the statistical significance of groups was analyzed using the Mann-Whitney U test, one-way ANOVA, Tukey's and least significant difference post hoc test (SPSS, Chicago, Ill., USA).

Differences were considered statistically significant when P<0.05, and statistical significance is expressed as follows: *P<0.05; **P<0.01; ***P<0.001.

### Example 4. Electrical and Optical Analysis of Non-Thermal Plasma (NTP)

### 4-1. Electrical and Optical Analysis of NTP

The optical emission spectra obtained to distinguish various excited plasmas produced by the N₂ plasma jet over a wide range of wavelengths (200 to 900 nm) are illustrated in FIG. 1B.

The emission spectra are manifested according to the presence of excited nitrogen, and may be divided into a N₂ secondary positive system, a N₂ primary positive system and a N₂ primary positive system in a range of 320 to 360 nm, 370 to 430 nm and 460 to 690 nm, respectively.

A strong NOγ band was detected at 200 to 271 nm, and a hydroxyl radical (A2Σ++X2Π) was also detected at 306 to 312 nm. Electrical characteristics were analyzed using a digital phosphor oscilloscope (DPO4054B, Tektronix, USA) to confirm the production of stable plasma. A general Pen type AC plasma jet applied in the present invention is driven under a frequency condition of several tens of kHz, and generates a minute electric charge at a half cycle of a sine wave

Referring to FIG. 1C, a single current peak is produced per half voltage wave when the voltage drops and the current increases. The root mean square values of voltage (Vrms) and current (Arms) are found to be 0.431 kV and 46.4 mA, respectively.

### 4-2. Confirmation of Generation of Active Species in Plasma Jet

A gas produced by plasma generation according to an exemplary embodiment of the present invention was analyzed, and it was confirmed through FIGS. 1D and 1E that O₃ and NOₓ were generated as by-products. The amount of ozone generated by the plasma jet was measured using an ozone monitor (106-M, 2B Technologies, USA) with a measurement error of 0.01 ppm, and the concentration of NOₓ produced by the plasma jet was measured using a portable gas analyzer (MK9000, ECOM, Germany) with a measurement error of ±0.5 ppm (open air). Oxygen in the atmosphere is decomposed and recombined under the influence of the nitrogen plasma jet to produce a small amount of ozone and produce UV. Referring to the graph in FIG. 1D, it can be confirmed that the increase in initial ozone concentration is due to the saturation time of the ozone analyzer, and the concentration of 9 ppm is maintained constant for about 20 minutes.

The amount of NO₂ produced by the plasma jet was confirmed to be 7.4 ppm on average (open air). Meanwhile, since the NO—NO₂ conversion proceeds as follows, it can be seen that a very small amount of NO was produced in the plasma jet, and it can be assumed that most of NO is converted into and produces NO₂ or O₃.

O₂+e→O+O+e (1)

NO+O→NO₂

2NO+O₂→2NO₂ (3)

O₂+O→O₃ (4)

NO+O₃→NO₂+O₂ (5)

### Example 5. Confirmation of the Angiogenesis-Promoting Effect of NTP

### Example 5-1. Confirmation of Angiogenesis-Promoting Effect of N₂ Non-Thermal Plasma Treated Solution (NTS)

Although nitric oxide produced by endothelial NO synthase (eNOS) has been reported to play an important role in vascular development and proliferation of endothelial cells, it is not clear how gas molecules differently regulate signals in cells according to intrinsic and extrinsic pathways.

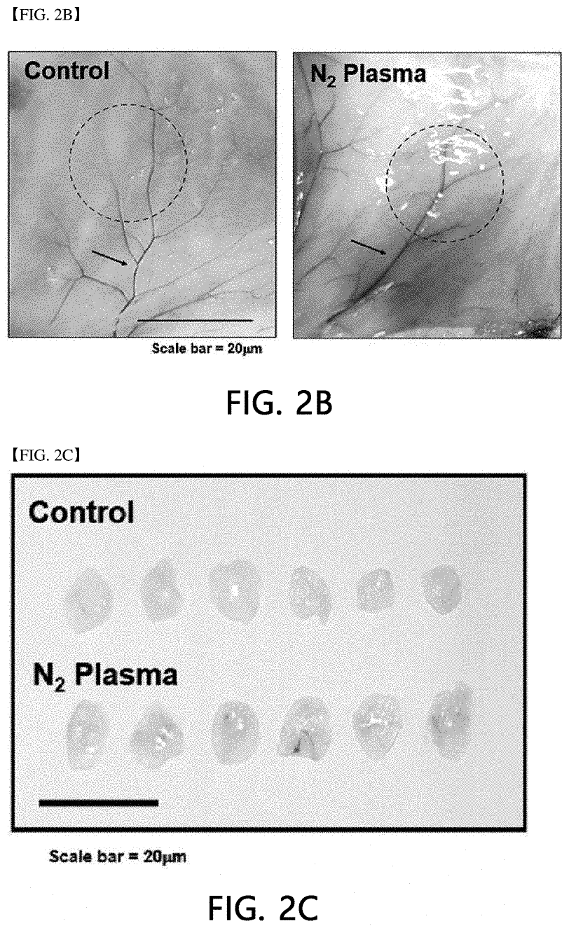

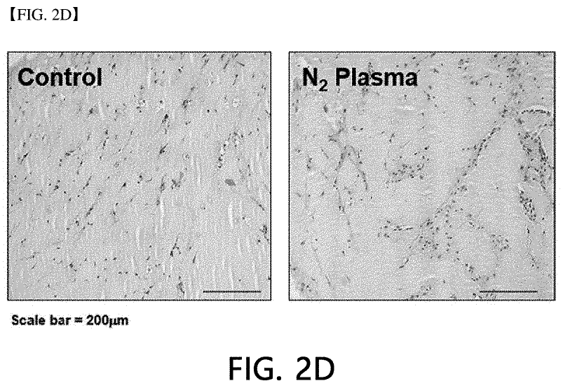

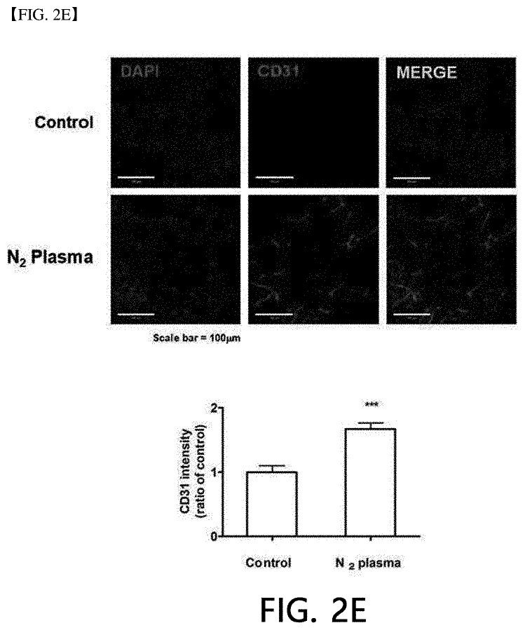

Therefore, the present inventors performed a Matrigel plug analysis for monitoring whether NTS increased vascular recruitment by treating the control and NTS. First, the present inventors confirmed whether NTS had a cytotoxic effect, and a mouse survival analysis showed that NTS was not cytotoxic (FIG. 2A). Referring to FIG. 2B, it can be seen that NTS treatment not only increased angiogenesis but also increased the thickness of blood vessels, and compared to the control, more red color was observed, indicating that there were more red blood cells in the newly formed vessels (FIG. 2C).

Furthermore, as a result of H&E staining analysis, cells migrated to the periphery of blood vessels in vivo in the NTS-treated group compared to the untreated group (FIG. 2D). Immunofluorescence stained with a CD31 antibody also showed the same pattern consistent with the sections of the Matrigel plug (FIG. 2E).

That is, it can be seen that the NTS prepared by the present invention promotes angiogenesis of endothelial cells in vivo.

### Example 5-2. Confirmation of Effect of NTS on eNOS-Activated Growth and Capillary Structure Formation in HUVECs

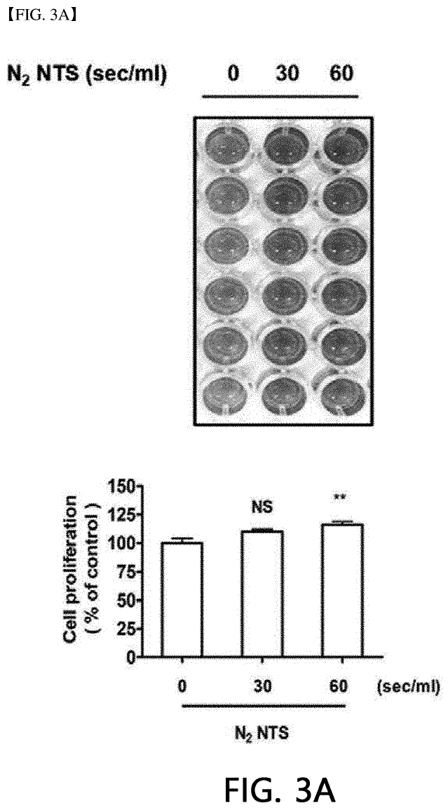

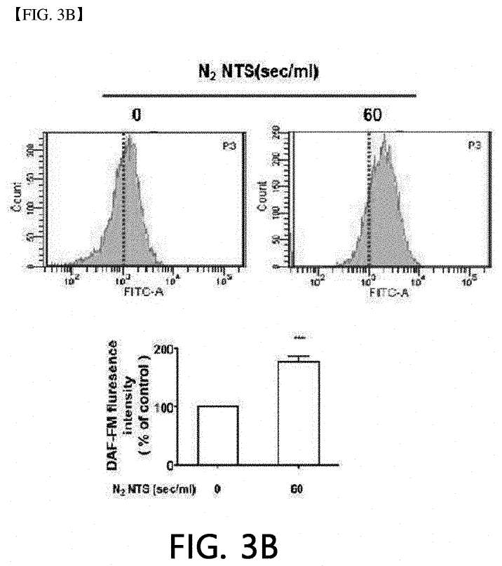

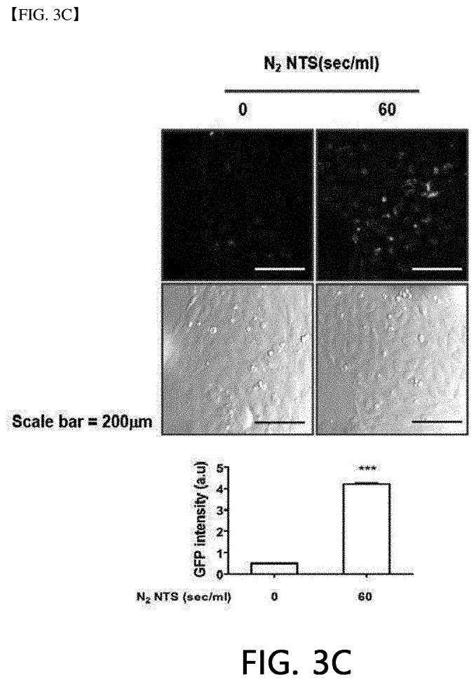

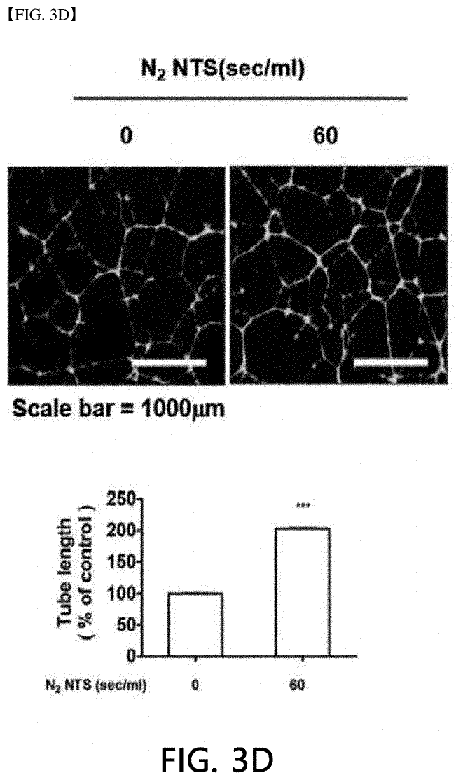

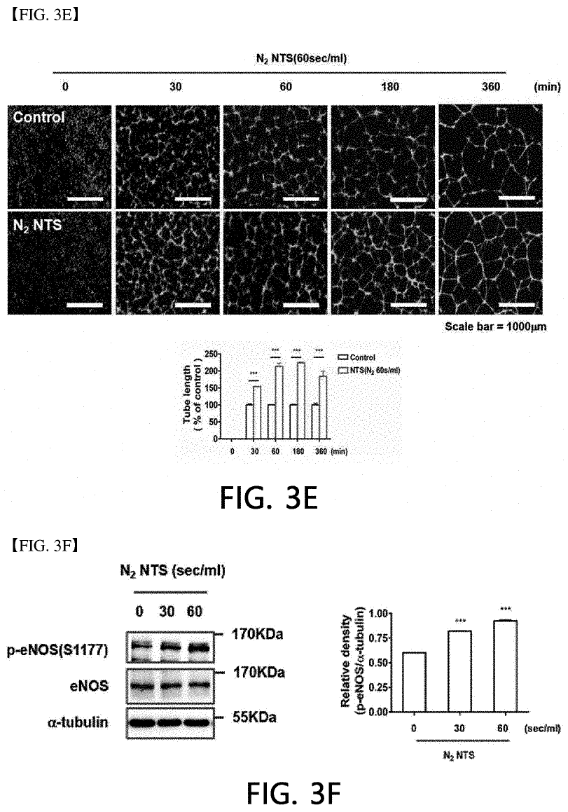

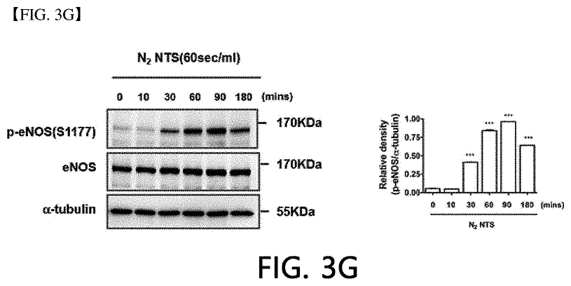

To investigate the effect of NTS on endothelial cells, HUVECs were treated with NTS several times (30, 60 sec/ml), and the effect on cell proliferation was analyzed by BrDu assay. As illustrated in FIG. 3, the extent of proliferation was increased significantly in proportion to the NTS treatment time.

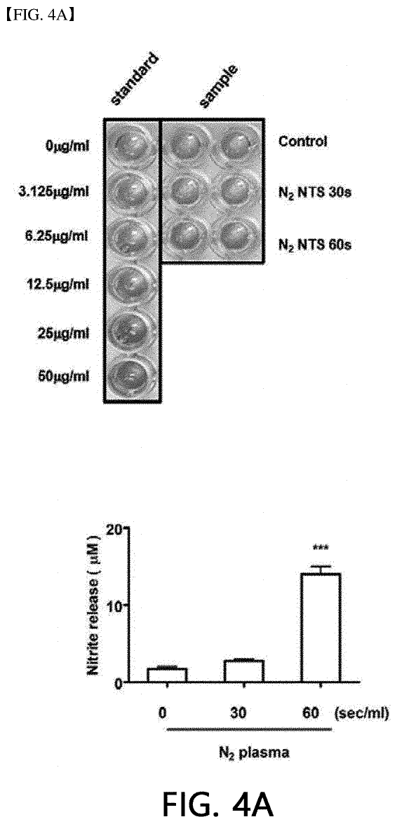

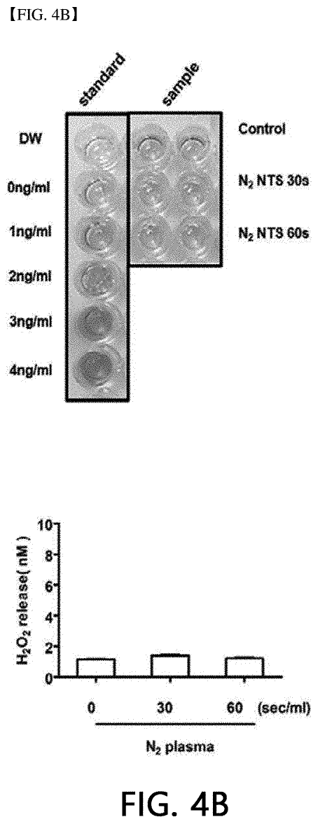

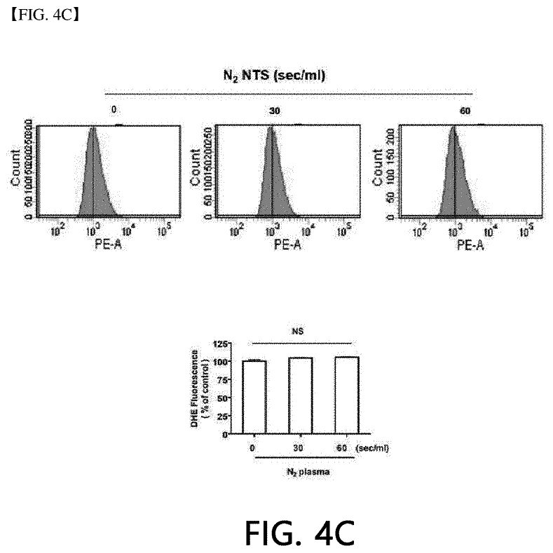

In FIG. 4A, the optical emission spectrum of the N₂ NTP shows that micro NTP jets produced exited nitrogen atoms after treatment.

It is well known that nitric oxide (NO) dilates blood vessels, regulates cell growth and maintains vascular homeostasis. Therefore, intracellular NO levels were determined using a fluorescent probe DAF-FM to see whether NO produced by NTS affects the activation of endothelial cells. The results from this showed that NTS induced intracellular NO in HUVECs particularly upon NTS treatment at 60 sec/ml (FIG. 4A). Interestingly, levels of extracellular nitric oxide (NO), which is an RNS, were also increased in the presence of NTS (FIG. 4A).

However, ROS levels were not significantly increased compared to NTS-untreated cells (FIGS. 4B and 4C). This result suggests that NTS activates endothelial cells by increasing NO levels.

Next, the effect of NTS treatment on pre-formed tubes for HUVECs supplemented with NTS (30, 60 sec/ml) after HUVECs had already formed tubular networks in Matrigel was confirmed. Quantification of tube length in this analysis showed that NTS treatment improved tube length by 1% (not significant) and 52% (p≤0.001) at 30 and 60 sec/ml, respectively (FIG. 3D).

Further, to determine the timing of the effect of NTS on tube formation, NTS-treated HUVECs were plated on Matrigel and tube formation was observed at 0, 1, 3 and 6 hours. As a result, a significant increase in endothelial tube formation was observed at 30 minutes of NTS treatment compared to the control, and the effect was more pronounced at 3 hours (FIG. 3E).

Studies in the related art confirmed that eNOS-derived NO also regulates inflammation, immune responses and angiogenesis. Therefore, the present inventors measured the expression of eNOS, which is a main enzyme responsible for NO production. Western blot analysis shows that NTS treatment significantly induces eNOS phosphorylation in HUVECs (FIG. 3F). Consistent with the above results, as a result of treatment with NTS at each time, the phosphorylation of eNOS was significantly induced between 1 and 3 hours (FIG. 3G).

All of the above results imply that the levels of intracellular nitric oxide become higher with NTS treatment and that eNOS activates growth in endothelial cells.

### Example 5-3. Confirmation of Effect of Increasing Cell Migration Through Extracellular Matrix (ECM) Activation According to NTS Treatment

Since the essential function of nitric oxide is to stimulate cell growth and migration by activating the eNOS signaling pathway in endothelial cells, it was evaluated whether NTS affects such processes.

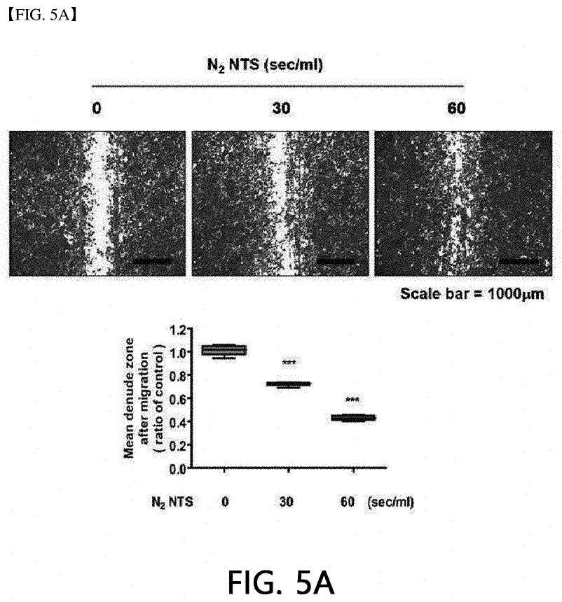

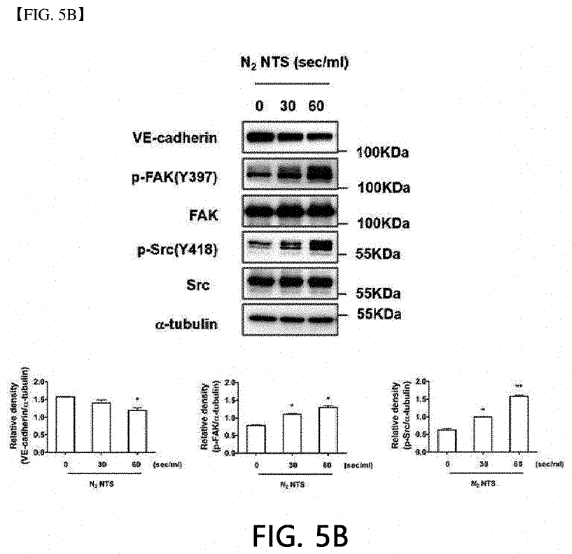

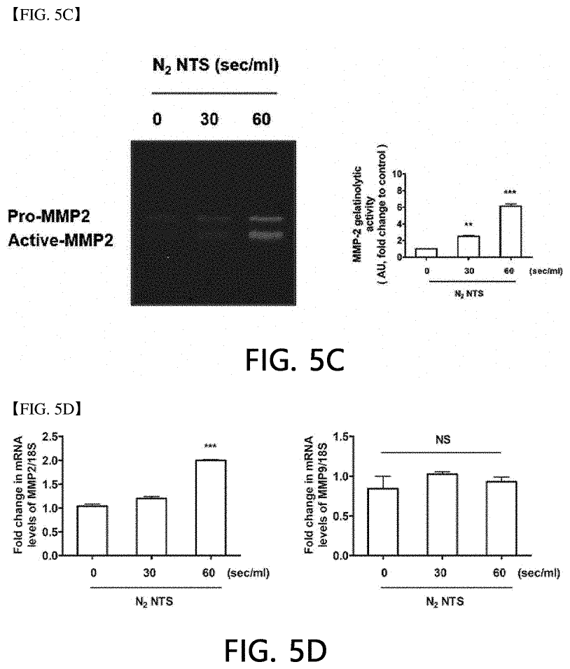

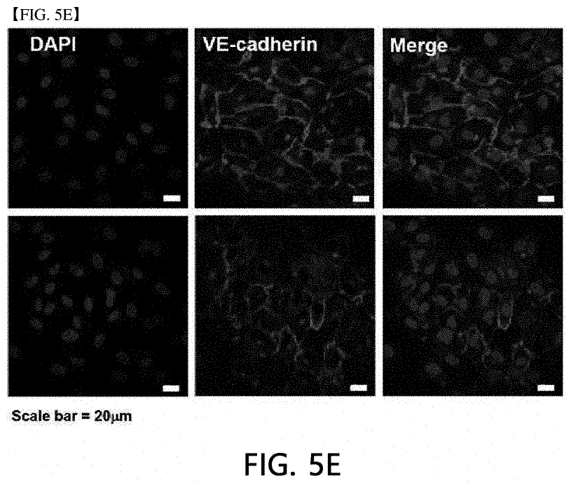

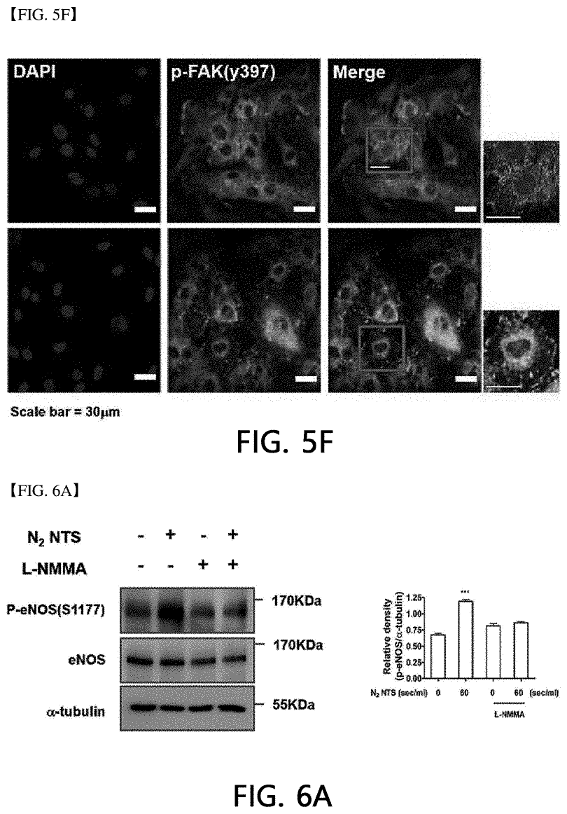

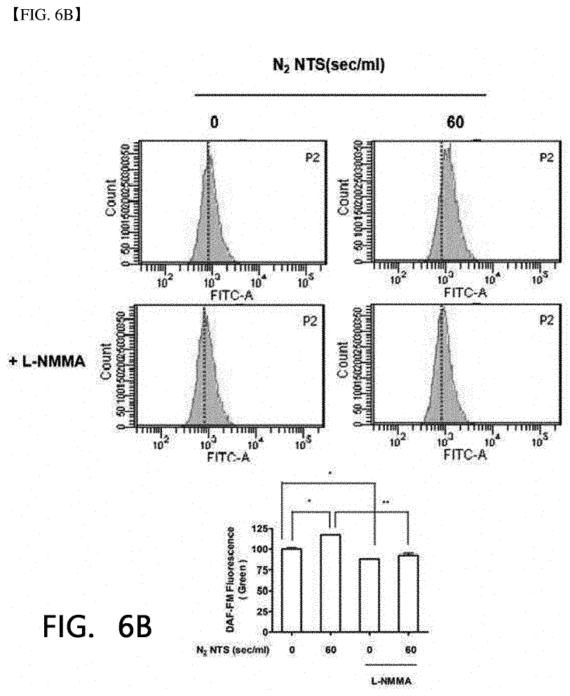

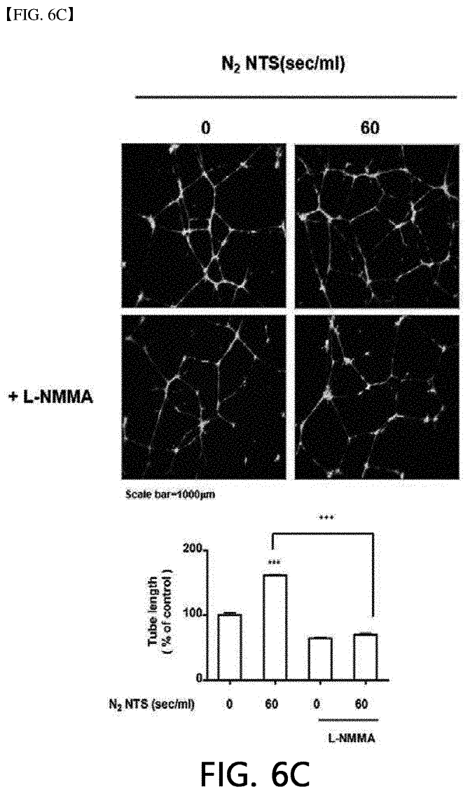

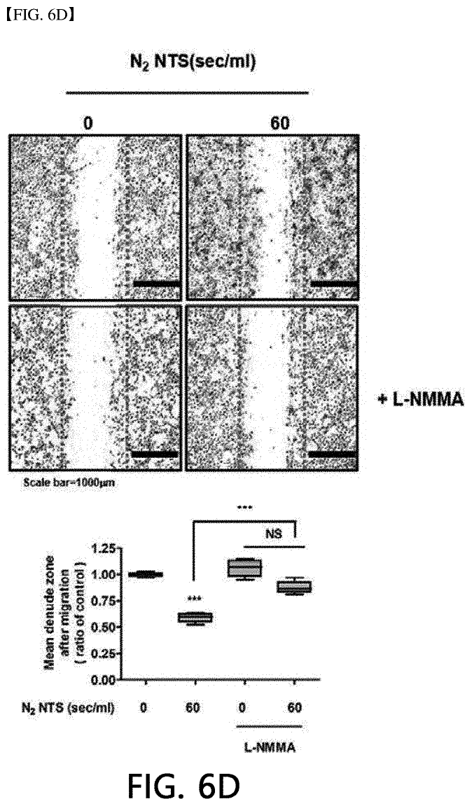

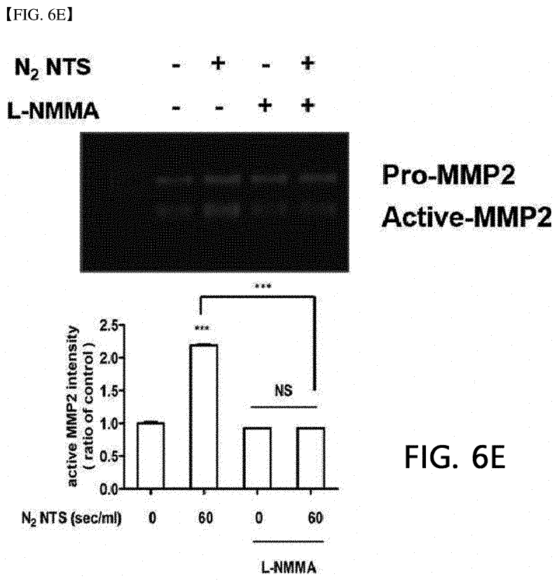

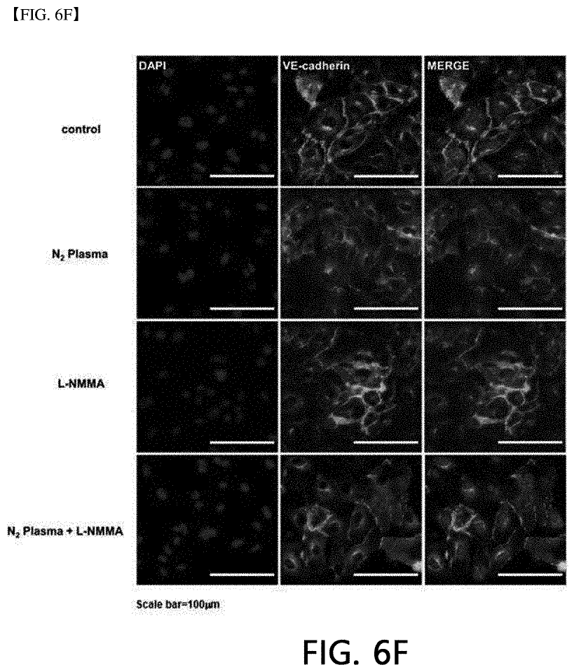

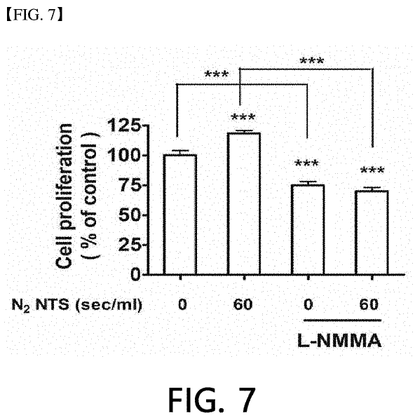

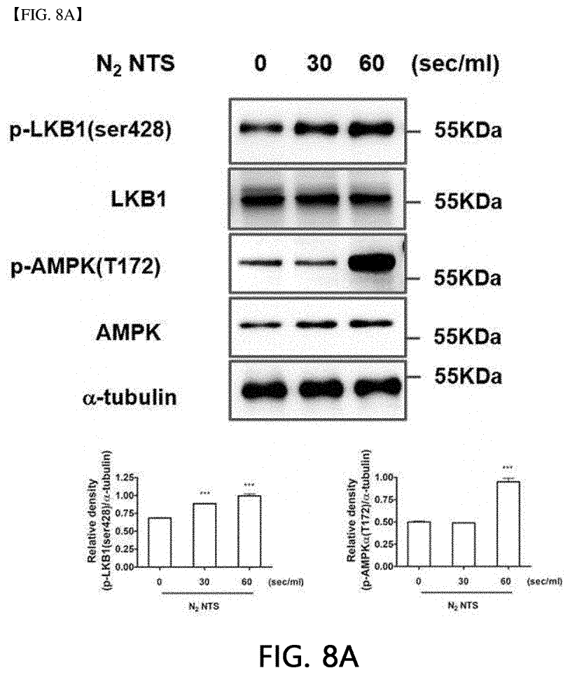

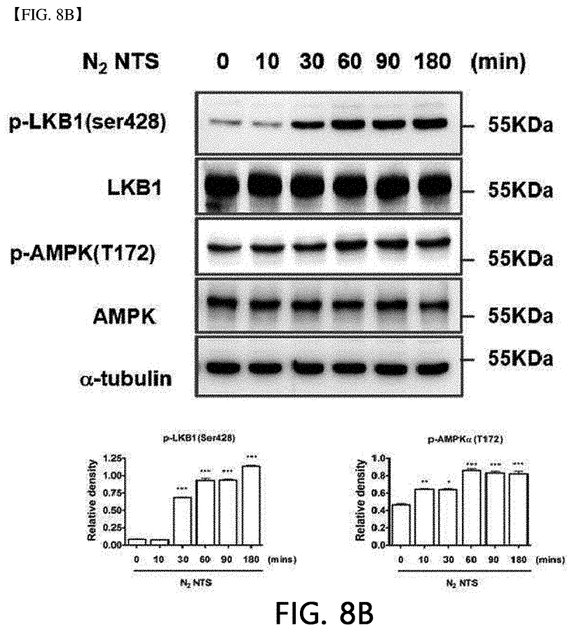

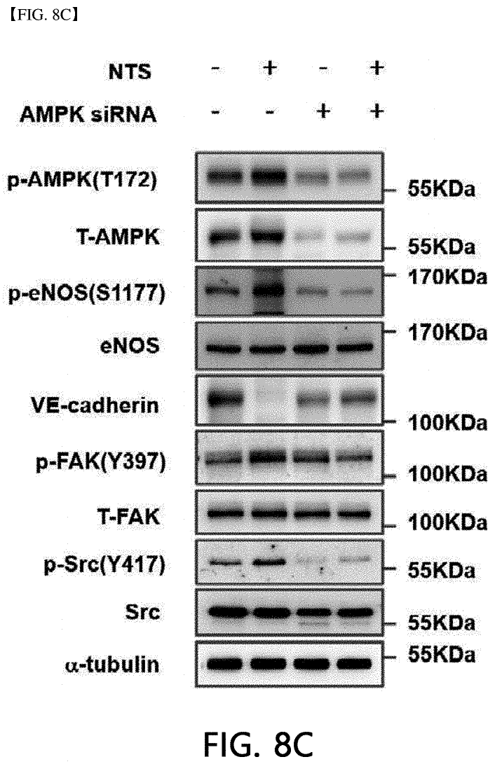

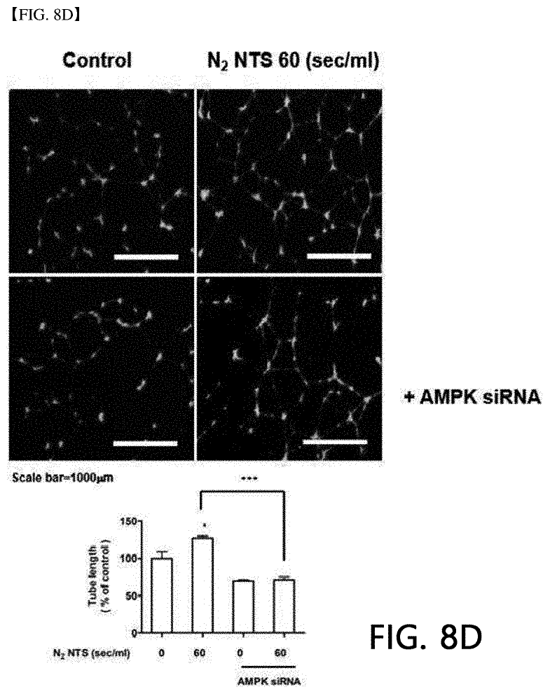

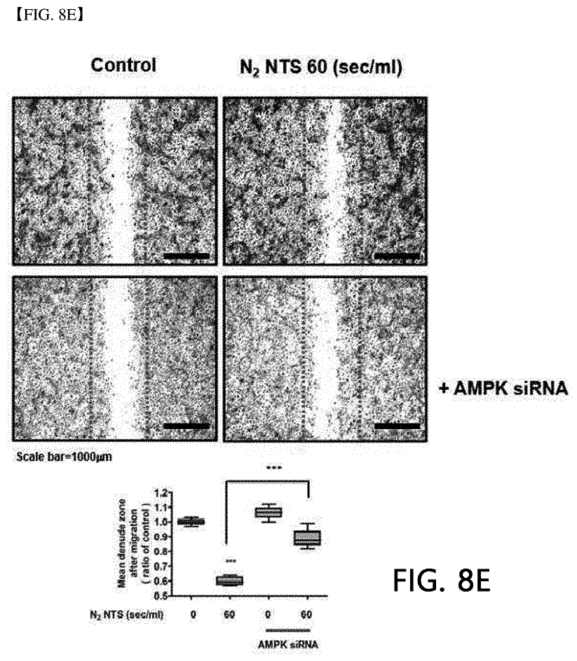

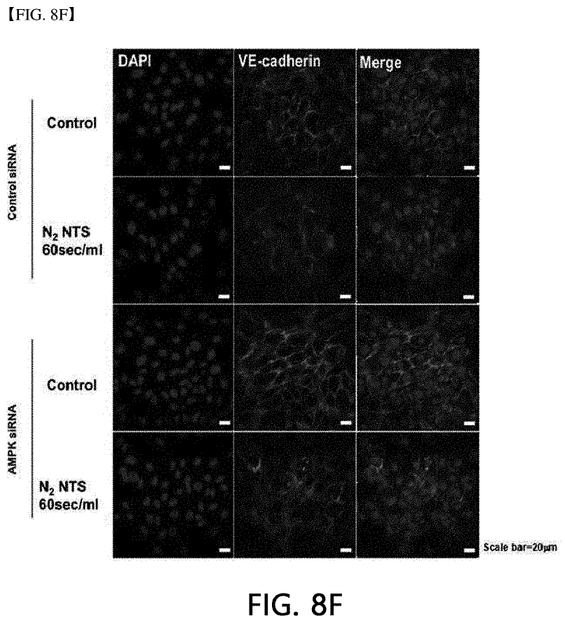

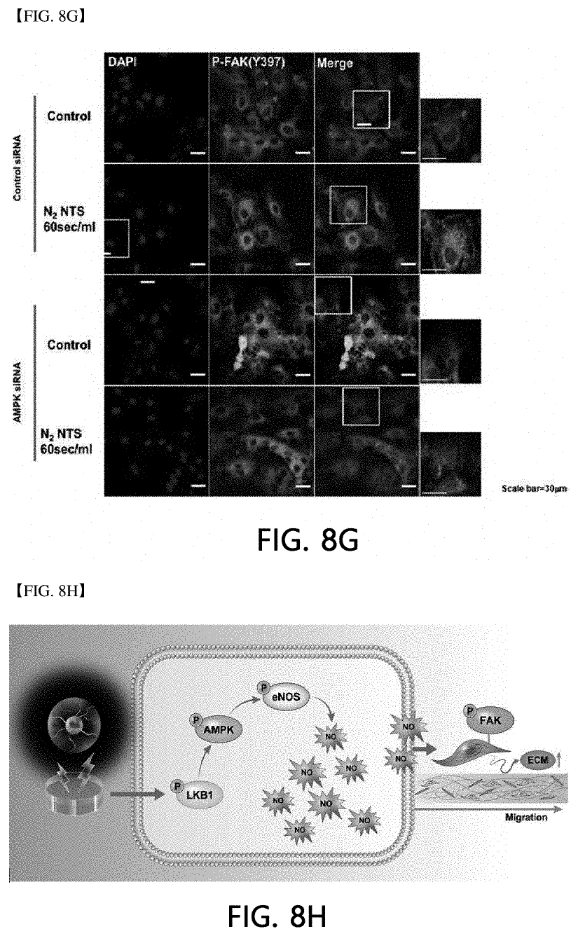

The present example evaluated whether nitric oxide activation of the eNOS signaling pathway, which stimulates cell growth and migration in endothelial cells, is affected by NTS. The corresponding results are illustrated in FIG. 5A. Referring to FIG. 5A, it can be seen that NTS treatment significantly increases the migration of HUVEC (P<0.001) cells across the denuded zone. The migration of HUVEC cells was increased by 34.2% and 58.7%, respectively, compared to the control when the HUVEC cells were cultured for 24 hours after NTS (30 sec/ml, 60 sec/ml) treatment.

In addition, the present inventors evaluated the effect of NTS on cell migration, and evaluated protein levels for VE-cadherin and phosphorylation of FAK (y397) and Src (y418), which are known to be closely associated with cell migration, invasion and cytoskeletal rearrangement through VE-cadherin and an FAK/Src kinase complex.

After NTS treatment, the present inventors confirmed an increase in FAK phosphorylation, a treatment time-dependent increase and a decrease in VE-cadherin in phosphorylation of Src downstream of FAK (FIG. 5B).

According to studies in the related art, it is known that a focal adhesion kinase (FAK) mediates cell matrix rearrangement processes by transmitting signals to a matrix metalloproteinase (MMP), which plays an important role in cell migration.

The present inventors used gelatin zymography for MMP-2 activity in order to confirm whether NTS induces MMP-2 activity to play an important role in cell migration. The corresponding results showed that MMP-2 activity was remarkably increased when HUVEC cells were treated with NTS at 30 sec/ml and 60 sec/ml, respectively, compared to the control group (FIG. 5C).

Furthermore, the present inventors evaluated the mRNA expression of MMP-2 using real time-PCR in order to additionally confirm the effect of NTS on MMP2. As illustrated in FIG. 5D, NTS treatment significantly increased MMP-2 mRNA expression.

Finally, the expression of VE-cadherin and p-FAK was confirmed again through immunofluorescence analysis (FIG. 5E). VE-cadherin staining showed inhibition of protein distribution among ECs in the NTS-treated group compared to the control (FIG. 5E). Meanwhile, the expression of p-FAK was clearly expressed in the NTS-treated group compared to the control (FIG. 5F).

According to the present example, it can be seen that NTS increased cell migration by increasing FAK signaling and MMP activity.

### Example 5-4. Via NTS LKB1 AMPK Signaling

To explain the underlying mechanisms of NTS migration and proliferation in endothelial cells, the present inventors evaluated the effect of NTS-induced changes on eNOS downstream signaling.

### Example 5-5. Plasma Increases Cell Migration and Tube Formation Through AMPK Signaling

The present inventors used L-NMMA (10 μM) to confirm whether NTS is sensitive to NOS inhibitors. L-NMMA significantly reduced p-eNOS phosphorylation and intracellular nitric oxide by NTS (FIGS. 6A and 6B). Even under these experimental conditions, L-NMMA treatment significantly disrupted a tubular network formed by NTS (FIG. 6C).

The present invention relates to a method for preventing or treating angiogenesis-related diseases using a liquid type plasma (a liquid composition treated with non-thermal plasma), and since the liquid type plasma of the present invention has a remarkable effect of promoting angiogenesis of blood vessels, the liquid type plasma of the present invention can be widely utilized in the prevention and treatment of related diseases.

What is claimed is:

1. A method for preparing a liquid type plasma for promoting angiogenesis, the method comprising: (a) filling a plasma generator with a carrier gas;
(b) generating plasma by supplying a voltage of 1 kV to 10 kV and a frequency of 10 to 30 kHz to the plasma generator; and
(c) irradiating a liquid material with the generated plasma.

2. The method of claim 1, wherein the carrier gas in Step (a) is any one or more selected from the group consisting of nitrogen, helium, argon, and oxygen.

3. The method of claim 1, wherein the irradiation in Step (c) is performed for 5 minutes to 120 minutes.

4. The method of claim 1, wherein the liquid material in Step (c) is water, saline, a buffer, or a medium.

5. A pharmaceutical composition for preventing or treating angiogenesis-related diseases, comprising a liquid type plasma prepared by the method of claim 1 as an active ingredient.

6. The pharmaceutical composition of claim 5, wherein the pharmaceutical composition is an oral formulation, a parenteral formulation or a topical formulation.

7. The pharmaceutical composition of claim 5, wherein the pharmaceutical composition is used alone or in combination with methods using surgery, radiation therapy, hormone therapy, chemotherapy and a biological response modifier.

8. The pharmaceutical composition of claim 5, wherein the angiogenesis-related disease is at least one disease selected from the group consisting of wounds, burns, varicose veins, ischemia, infertility, diabetic foot ulcers, ischemic stroke, ulcers, arteriosclerosis, myocardial infarction, angina pectoris, ischemic heart failure, bedsores, alopecia and cerebrovascular dementia.

9. A method for preventing or treating angiogenesis-related diseases, the method comprising administering a liquid type plasma prepared by the method of claim 1 to a subject other than a human.

10. The method of claim 9, wherein the angiogenesis-related disease is at least one disease selected from the group consisting of wounds, burns, varicose veins, ischemia, infertility, diabetic foot ulcers, ischemic stroke, ulcers, arteriosclerosis, myocardial infarction, angina pectoris, ischemic heart failure, bedsores, alopecia and cerebrovascular dementia.

11. A use of a pharmaceutical composition comprising a liquid type plasma prepared by the method of claim 1 as an active ingredient for preventing or treating angiogenesis-related diseases.

12. The use of claim 11, wherein the pharmaceutical composition is an oral formulation, a parenteral formulation or a topical formulation.

13. The method of claim 11, wherein the pharmaceutical composition is used alone or in combination with methods using surgery, radiation therapy, hormone therapy, chemotherapy and a biological response modifier.

14. The use of claim 11, wherein the angiogenesis-related disease is at least one disease selected from the group consisting of wounds, burns, varicose veins, ischemia, infertility, diabetic foot ulcers, ischemic stroke, ulcers, arteriosclerosis, myocardial infarction, angina pectoris, ischemic heart failure, bedsores, alopecia and cerebrovascular dementia.
